(19) United States
(12) Patent Application Publication
(10) Pub. No.: US 2007/0249994 A1
(43) Pub. Date: Oct. 25, 2007

(54) Mixing and injection system for injectable biomaterials or artificial materials in orthopaedic applications

(76) Inventors: Thomas Uhlin, Uppsala (SE); Tobias Persson, Uppsala (SE); Erik Olaisson, Uppsala (SE)

Correspondence Address: WIGGIN AND DANA LLP;ATTENTION: PATENT DOCKETING, ONE CENTURY TOWER, P.O. BOX 1832, NEW HAVEN, CT, 06508-1832, US

(21) Appl. No.: 11/725,412

(22) Filed: Mar. 19, 2007

Related U.S. Application Data
(60) Provisional application No. 60/784,085, filed on Mar. 20, 2006.

(51) Int. Cl. A61M 5/19

(52) U.S. Cl. 604/82; 604/518

(57) ABSTRACT

The present invention specifically relates to a mixing and injection device for mixing, handling and injecting of biomaterials, such as ceramic biomaterials, PMMA and other materials intended for stabilising and healing fractures and lesions, and for filling voids in orthopaedic, spinal, cranio-maxillofacial, distal radius, tibia plateau or other applications where natural or artificial substances are to be delivered to such fractures, lesions or voids in the skeleton.

### CROSS-REFERENCE TO RELATED APPLICATIONS

This application claims the benefit of U.S. Provisional Patent Application Ser. No. 60/784,085 filed Mar. 20, 2007.

### BACKGROUND OF THE INVENTION

1. Field of the Invention

This invention refers to a system for mixing, handling and injecting of injectable materials, in particular injectable biomaterials, such as injectable biocompatible ceramic materials, or PMMA and other natural or artificial materials intended for stabilising and healing fractures and lesions, and for filling voids in orthopaedic, spinal, cranio-maxillofacial, distal radius, tibia plateau or other applications where said materials are to be delivered to such fractures, lesions or voids in the skeleton. The present invention specifically relates to a mixing and injection system for orthopaedic applications, in particular VertebroPlasty (PVP), and also to a method of mixing and injecting a biomaterial using said mixing and injection system.

2. Description of the Related Art

Proper mixing, handling and injection of ceramics, resins and other biomaterials are crucial for obtaining an excellent result, since many compositions are not easily mixable, introduction of infection should be avoided and injection should be performed quickly and with precision. If radiographic imaging is used during the injection, then the operator should preferably be able to control the delivery from outside the field of radiation.

Injectable materials, such as bioceramics and resin-based compositions, e.g. PMMA, are traditionally accompanied by a variety of procedures and tools for mixing, handling and injection. Since different materials for orthopaedic use have different features they are not easily compared. Some of the materials are easy to mix, for example PMMA. PMMA is normally mixed in an open vessel using a spatula. By using an open vessel there is always a small risk of introducing infectious material into the material and the user is on a daily basis exposed to toxic fumes. There are also closed vessels for PMMA in which a stirring paddle is used for mixing purposes.

A general problem with PMMA mixing and delivery is that once the material is mixed, it has initially very low viscosity, and if injected at this stage, risks leakage to not intended areas in the body. If, on the other hand, the operator waits until the material has higher viscosity and the risk of leakage is lower, then the working time is very short as the hardening of PMMA occurs exponentially.

Some resin-based materials are mixed and delivered through a double-barrel syringe where mixing is accomplished in the tip (nozzle) of the syringe, which is a static mixer. The advantage of this system is that hardening only starts after the components in the respective barrels meet in the tip. This extends the time available for working with the material before it hardens. However, in this case effective mixing demands low viscosity, it requires that the content in the two barrels exhibit low viscosity, and consequently the viscosity will also be low in the material that is extruded out of the tip, leading to potential concerns about in-vivo leakage. Many physicians use standard syringes, e.g. 1 cc and 2 cc syringes to inject the material. This requires a fairly time-consuming preparation to place the material in the syringes.

Other delivery systems are based on high pressure and allow the operator to control the system without being exposed to radiation as the material is injected through a catheter of adequate length. The principles for applying high pressure and the ergonomics of some of these systems are not optimal. Also, they provide sub-optimal the control of the amount of material injected is not optimised.

Ceramic bone replacement materials can have important advantages to resin-based materials, e.g. in their safety, efficacy and handling. They typically, however, require a higher amount of energy in the mixing step to get homogeneous cement, which is important for the performance of the material. For some ceramic material, it is still possible to mix them by hand with a cup and spatula or a disposable plastic mixer. Other ceramic materials are mixed in a rubber balloon in which the material components are placed and the composition is massaged by hand. Some ceramic materials require vigorous mixing, which may be done performed most effectively by using an automatic shaking/vibrating apparatus.

### SUMMARY OF THE INVENTION

The object of the present invention is to eliminate the drawbacks of the prior art techniques and devices for mixing and injecting orthopaedic materials and to provide a mixing and injection system suitable for injectable ceramic biomaterials or other materials, intended for orthopaedic use, and which achieves robust and reproducible mixing, minimises the risk of introducing infection, minimises the exposure to radiation and toxic fumes during handling and treatment, and improves control, precision and safety during the injection.

The present invention achieves this by a mixing and injection system for mixing and injecting biocompatible ceramic materials or an artificial material, or combinations, into the skeleton, in particular the vertebrae, of a human or animal subject.

According to a first aspect, the present invention provides a mixing and injecting device. In a basic embodiment, said mixing and injector device comprises an injector device, comprising a container, said container comprising a first component (in particular a ceramic powder, or a component of a material such as PMMA) and a second component (a component initiating curing of the first component, such as a hydration liquid if a ceramic powder constitutes the first component), separated from first component, a grip, and means for feeding the mixture of said components out of the container.

Said injector device also be suitable for other injectable non-ceramic biomaterials such as for example PMMA, if they have a viscosity that is suitable for injection using said injecting device or system. However, for the purpose of simplifying the present description, the mixing and injection system as well as the injector device is only described featuring ceramic materials (in this case a hydrating ceramic material) and a hydration liquid. However, if used with for example PMMA (or any other biomaterial), the operation of the system and device is similar, but one of the PMMA components may replace the ceramic powder material, and the other may replace the hydration liquid.

The injector device may partly or fully be enclosed in a blister pack or other primary package, such that the sterility requirements may be assured.

Said injector may further comprise extension tubes for enabling injection at a distance from a patient to be treated. This provides the opportunity of being outside the field of radiation during simultaneous radiation treatment.

Said injector device may also be combined with a hydraulic means for pushing the ceramic composition or material out of the container. Said hydraulic means being connected to the proximal end of the container.

The injector device imparts sufficient force transmission to enable injection from a container/syringe of 10 ml. The injector is also designed to have a “dead man's handle”, i.e. when the grip is released, almost no cement is injected. The protruding means on the arms of the grip is designed to feed the force straight forward in the direction of the syringe, thereby avoiding the teeth jumping out of the position in the grooves/notches of the piston. The design of the grip also enables normal pace-work, whereas many prior art constructions require “working in slow motion” in order to inject in a proper and safe way. The design of the grip is also such that it gives the injecting person the right transmission to enable safe and easy injection into for example a spine, and which also enables repetitive squeezing of the grip.

The double piston construction of the injection system (including the hydraulic extension) prevents any hydraulic fluid from entering the container of the injectable biomaterial material, thereby eliminating any contamination of said material during the injection.

According to a second aspect, the present invention provides a method using the injector device according to the present invention. A basic embodiment of said method comprises the steps of:

mixing the a first component of the biomaterial and second component initiating curing of said first component (e.g. a hydraulic ceramic powder and hydration liquid, respectively, or a PMMA base material and a component initiating curing of the PMMA base material);

optionally shaking said mixture (by for example a shaking or vibrating apparatus);

transferring the biomaterial to a suitable container (syringe) for injection, and

inserting a plunger into the container (syringe) and inserting the container and plunger into the injector device according to the invention, or inserting the container (syringe) into the injection system,

feeding the biomaterial out of the container using the injector device or injection system, and injecting said biomaterial into a designated void, for example a space between two vertebrae of a spine.

According to a third aspect, the present invention provides a transfer device suitable for transferring a biomaterial to an injector device or injection system according to the present invention. The transfer device comprises a protective cover that keeps the biomaterial sterile during the filling of a single-chamber container (syringe) and protected when used in a non-sterile zone, for example in an operating room. The protective cover and the single-chamber container (syringe) may then easily be removed together, without touching the syringe, and transferred to a sterile zone. The syringe may then be removed in the sterile zone.

This invention presents an alternative, and improved, way of mixing, handling and injecting a biocompatible or a natural or artificial material, or combinations thereof, into the skeleton, in particular the vertebrae, of a human or animal subject. It presents a unique, closed, all-in-one system, comprising in one embodiment a sterile primary package that allows mixing of solution and powder without breaking the sterile barrier. In addition, it can directly be used with an electric vibrating/shaking apparatus for robust and reproducible mixing. The uniquely designed grip then allows the mixed material to be safely and controllably injected. The injector may also be attached directly an injection needle/trocar for injection. The grip of the device injector may also be used outside the radiation using an extension system that allows the user excellent control of the injection/application outside of the radiation (from the CT, Fluoroscopy or other X-ray technique). Thus, a significant improvement in work flow and safety for the user is accomplished.

The invention is primarily designed to meet the demands of Percutaneous VertebroPlasty (PVP) used to treat vertebral compression fractures in patients with osteoporosis. This invention may also be applied for mixing, handling and injecting of biocompatible ceramic biomaterials, PMMA and other biomaterials intended for stabilising and healing fractures and lesions, and for filling voids in orthopaedic, spinal, cranio-maxillofacial, distal radius, tibia plateau or other applications where natural or artificial materials are to be delivered to such fractures, lesions or voids in the skeleton.

### BRIEF DESCRIPTION OF THE DRAWINGS

FIG. 1 is a perspective view of the injector device comprising the single-chamber container with a single plunger.

FIG. 2 is a perspective view of the injector device comprising the dual-chamber container with double plungers, where the inner plunger has been pushed into the outer plunger and broken the membrane separating the two chambers.

FIG. 3 is a perspective view of the injector device according to FIG. 2, where the outer plunger is screwed into the dual-chamber container, thus exposing the piston of the inner plunger.

FIG. 4 is a perspective view of the injector device according to FIG. 3, where the outer plunger is at a distal position and arms of the grip are pushed together such that the protruding member pushes the inner plunger into the outer plunger and thus the content out of the container.

FIG. 5 is a perspective view of the injector device according to FIG. 4, where the injector is connected at its distal end to a tube with an injection needle, for performing the injection into the vertebrae at a distance from a patient.

FIG. 6 is a perspective view of an injection system where the grip and the container comprising the material to be injected are separated by a hydraulic means with which the grip is used, for performing the injection into the vertebrae at a distance from a patient and with a power leverage.

FIG. 7 is a perspective view of the injection system according to FIG. 6, where the injector device according to FIG. 2, in this case comprising the single-chamber container, is connected to a cannula and is about to be connected to the coupling means of the hydraulic means.

FIG. 8 is a perspective view of the injection system according to FIG. 6, where the injector device according to FIG. 2, in this case comprising the dual-chamber container, is about to be connected to the coupling means of the hydraulic means.

FIG. 9 is a perspective view of a slightly different embodiment of the injection system according to FIG. 6.

FIG. 10 is a perspective view of a transfer device for transferring a pre-mixed biomaterial to a container to be inserted into the injection device or injection system according to the invention.

### DETAILED DESCRIPTION OF THE INVENTION

According to a first aspect, the present invention provides an injector system for ceramic biomaterials, such as for example Xeraspine™ or other ceramic bone cements. It is also applicable on other biomaterials, such as PMMA, calcium aluminates, and calcium phosphates. In a basic embodiment, said injector system comprises an injector device, comprising a container, said container comprising an injectable biomaterial, a grip, and means for feeding the injectable biomaterial out of the container.

Said basic embodiment comprises two main embodiments. In a first embodiment, i.e. embodiment 1, the container filled with the biomaterial is arranged in the grip of the injector device and the feeding means are arranged on the grip. In a second embodiment, i.e. embodiment 2, the container filled with the biomaterial is separated from the grip of the injector device by a hydraulic extension means.

According to embodiment 1A, said injector device comprises an injector device 1, comprising a container 2, said container comprising a ceramic powder and a liquid chamber, said container being arranged and secured by fastening means 3 at the distal end (may also be secured at other points in other embodiments) of a grip 4 having at its proximal end a means for feeding a plunger 5 into said proximal end of the container and pushing the ceramic composition or material out of the container via its distal outlet 2a.

Said grip 4 may comprise one or more arms 40a. Said arms may also comprise a protruding member 40b, arranged at the proximal end of the one or more arms 40a, and forming an angle of less than 90 degrees to said arms. Said protruding member may be flexible, rigid or be pivoted or hinged. The feeding means may be constituted by said arms and protruding member.

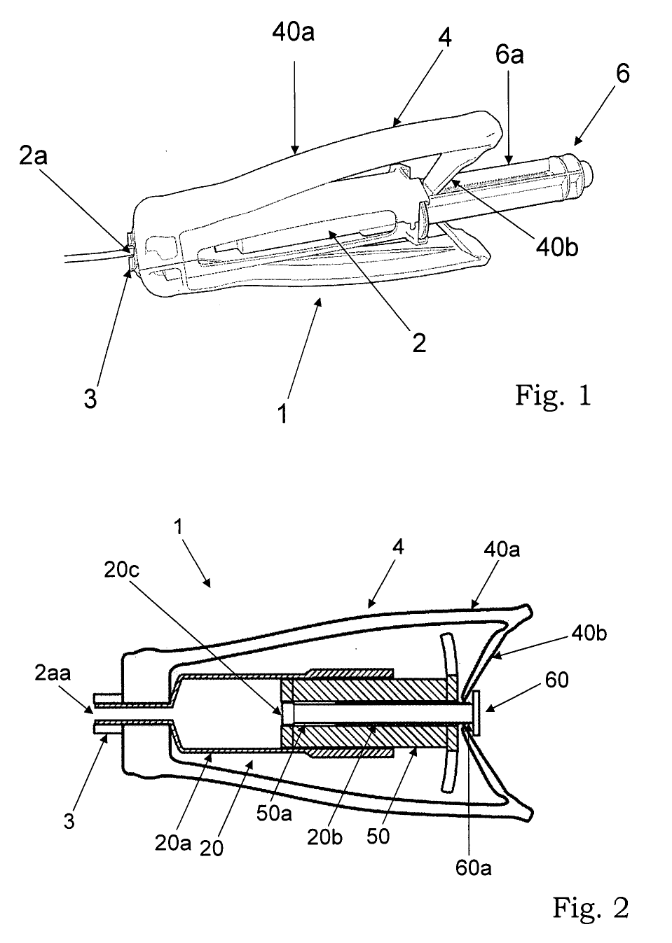

In a specific embodiment of said embodiment 1A, said injector device comprises a single-chamber container 2, said container comprising a pre-mixed ceramic composition or material, and having a distally located outlet 2aa, said container being arranged and secured by fastening means 3 at the distal end of a grip 4 having one or more arms 40a provided at the proximal end with a protruding member 40b forming an angle of less than 90 degrees to the arms of the grip, a plunger 6 (corresponding to the plunger 5 in the basic embodiment) partially arranged in the chamber, the piston 6a of which comprising notches or grooves. (see FIG. 1).

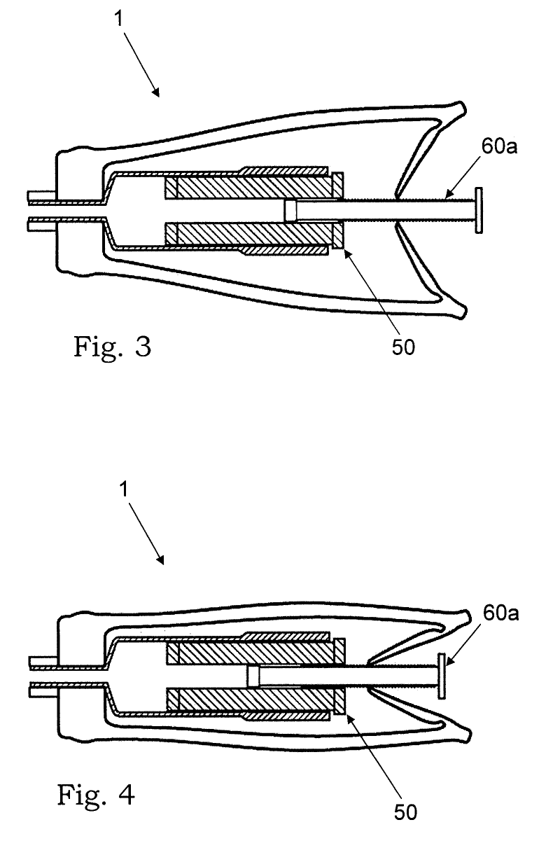

In alternative embodiment of embodiment 1A, i.e. embodiment 1B, the container 2 is a dual-chamber container 20 (comprising a ceramic powder chamber 20a and a liquid chamber 20b, occupying the distal and proximal part of the container, respectively) where the ceramic material and liquid are separated by a membrane 20c. Said alternative embodiment may also comprise two plungers (corresponding to the plunger 5 in the basic embodiment), an outer plunger 50 threaded into the proximal end of the container 20 and having a central bore 50a, and an inner plunger 60 partially arranged inside said central bore 50a (see FIG. 2). The distal part of the central bore which is not occupied by the inner plunger, initially forms a part of the liquid chamber 20b (can be seen in FIGS. 2-3).

In a specific embodiment of said embodiment 1B, said injector device comprises a dual-chamber container 20, said container comprising a ceramic powder chamber 20a having a distally located outlet 2aa and a liquid chamber 20b, said chambers being separated by a membrane 20c, said container being arranged and secured by fastening means 3 at the distal end of a grip 4 having one or more arms 40a provided at the proximal end with a protruding member 40b forming an angle of less than 90 degrees to the arms of the grip, an outer plunger 50 threaded into the proximal end of the container 20 and having a central bore 50a inside which an inner plunger 60 is partially arranged, the piston 60a of which comprising notches or grooves, the distal part of the central bore which is not occupied by the inner plunger initially forms a part of the liquid chamber 20b (see FIGS. 2-4).

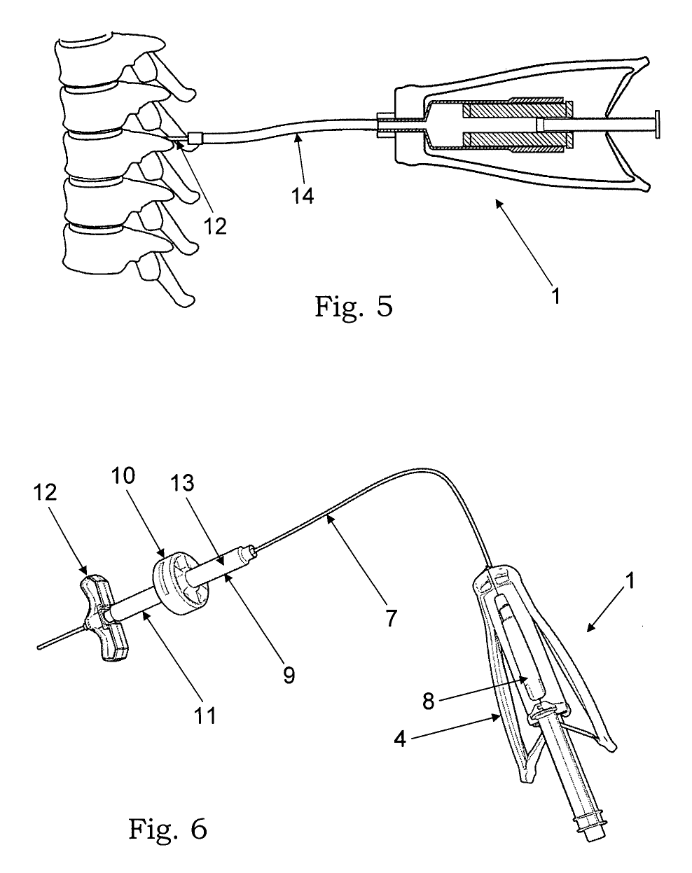

Embodiments 1A and 1B may also be combined with an extension tubing 14 to enable injecting from a distance from a patient. The extension tubing may be equipped with an injection needle/cannula 12 or a means for connecting to such a needle/cannula. (see FIG. 5).

As mentioned above, in embodiment 2 the container is separated from the grip and feeding means by a hydraulic extension means 7. The grip 4 may be one part of the hydraulic extension means. The grip and feedings means may be those described in embodiment 1A. The container filled with ceramic composition or material take the position of a third container 11, as described below.

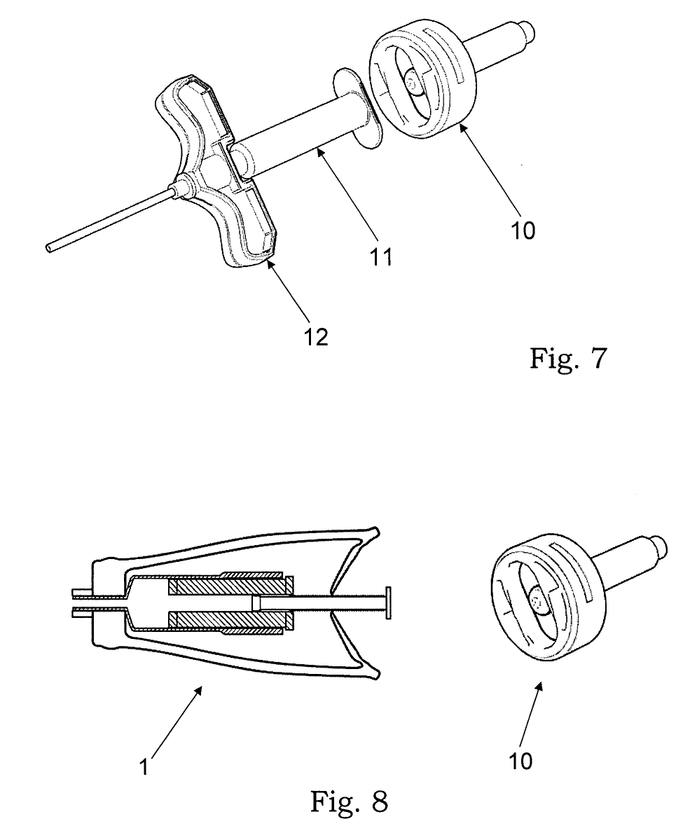

In a specific embodiment, the hydraulic extension means 7 is connected to the distal end of a liquid-filled container 8, and being in open contact with each other. Said container 8 is arranged in grip 4 and is arranged as described for the injector device in embodiment 1A and FIG. 1. Said hydraulic means may be a liquid-filled tube, and such as a water-filled tube. The hydraulic tube may in the distal end be connected to a second container 9 secured in a coupling means 10, which at its distal part is connected to a third container 11. The containers 9,11 and coupling means 10 form an open container into which a double-sided plunger 13 is positioned, said double-sided plunger 13 being in operative connection with the plunger of said container. The distal part of the third container 11 may be adapted to be secured in a cannula 12 by a Luer connector or the like. This also applies for the interconnection of the two containers in the coupling means 10, which may take the form of syringes. All of these components form an injection system. (see FIGS. 6-7).

According to embodiment 2A, said third container 11 is a single-chamber container as described in embodiment 1 and FIG. 1, which container has been filled with a ceramic powder and liquid, mixed in a shaking/vibrating apparatus, such that a ceramic composition or material has been formed. It is the prepared container which is secured in the connection means and cannula, as described above.

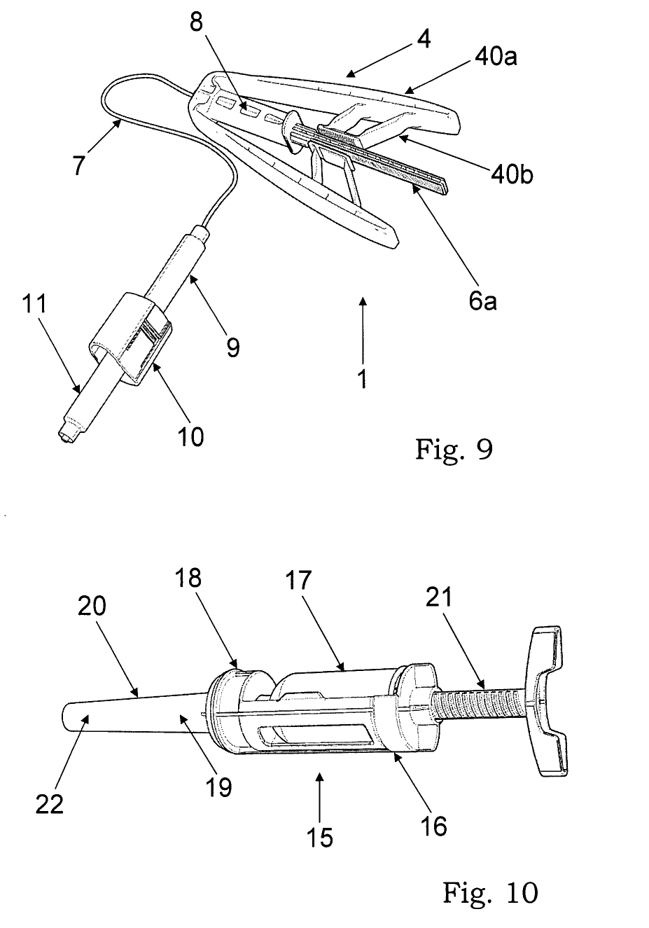

According to embodiment 2AA, which is a variant of embodiment 2A, the protruding member 40b on each arm 40a is split up into two protruding members that are integrally joined by a dented bridge, the teeth of which are in operative connection with the notches or grooves of the piston 6a. Otherwise, the function and the operation of the injector devices according to embodiments 2A and 2AA are the same. The injection system according to embodiment 2AA is depicted in FIG. 9.

According to embodiment 2B, said third container 11 is a dual-chamber, container as described in FIG. 2, initially comprising a ceramic powder and liquid separated by a membrane, which membrane has been broken, and the membrane is broken as described above by action of the inner plunger, the content of the container mixed in a shaking/vibrating apparatus, such that a ceramic composition or material has been formed. It is the prepared container which is secured in the connection means and cannula, as suggested above (see FIG. 8).

The containers used in the embodiments above may take the form of a syringe.

The grip and feeding means do not have to be integrated, but may also take the form of a grip and a separate feeding means, such as a screw, crank or handle.

The notches or grooves on the inner plunger may be any similar means interacting with the feeding means such that the plunger may be fed distally.

The dual-chamber container may be enclosed in a blister pack, or a similar sterile package, through all operations up to the mounting in the injector device or injection system. In the case of the single-chamber container, the ceramic material and the liquid may be enclosed and stored in blister packs or similar primary packages up to the moment they are to be mixed.

In embodiments 1B and 2B, the dual-chamber containers may be provided as syringes pre-filled with the ceramic powder liquid separated by a membrane. In embodiments 1A and 2A, the single-chamber containers may be provided as syringes pre-filled with the ceramic material or composition.

The filling of the single-chamber container, as described in embodiments 1A, 2A and 2AA, with a pre-mixed bone cement or biomaterial, may be performed by using a transfer device that assures that the mixing and transfer of the material to the single-chamber container is performed in a sterile and quick way (in order to meet the medical requirements and to avoid pre-mature curing). The present provides such a transfer device.

The transfer device is depicted in FIG. 10, wherein reference number 15 denotes said transfer device, which comprises a capsule holder 16, holding a capsule 17 which is compressible, or a capsule whose bottom can be pushed inside the body of the capsule or said capsule, containing a pre-mixed bone cement or biomaterial, a connector 18, which is connected to the distal end of the capsule holder 16 as a separate part, or forms an integral end part of said capsule holder, wherein said connector 18 connects the capsule 17 at it proximal end and a single-chamber container 19 at its distal end, such that an open chamber is formed between said capsule and single-chamber container, wherein said connector 18, at its distal part also connects a protective cover 20, said protective cover covering the open chamber, and wherein the transfer of the pre-mixed bone cement or biomaterial from the capsule to the single-chamber container is performed by a screw or crank 21, threaded into the proximal part of said capsule holder 16, which when operated (i.e. screwed), pushes in the bottom of the capsule 17 or compresses the capsule 17, such that its content is pushed forward and out of the capsule and into the single-chamber container 19. In order to remove the filled container, the protective cover 20 is disconnected from the connector 18 together with the filled container 19, and transferred to a sterile zone. In said sterile zone, the filled container may then be removed. The container may then be connected to the injection device according to embodiments 1A, 2A or 2AA before connected to the cannula positioned in the patients vertebrae for injection.

In embodiment 1A, the filled container 19 is inserted into the position of the container denoted by reference number 2 in the injector device described above and in FIG. 1. The container is thus secured in the grip 4 and a plunger, such as the plunger 6 having notches or grooves on its piston 6a, is inserted into the container. When correctly inserted, the protruding members 40b fit into the notches or grooves of the piston. Thus, when the arms of the grip is pushed together, the plunger is pushed into the container and the material is pushed out of the container via the needle/cannula. Said single-chamber container may optionally be attached to a short tubing 22 (elbow connector) that allows a vertical injection position during injection. Said short tubing (elbow connector) may be attached to the container before or after the filling step.

In embodiment 2A or 2AA, the filled container 19 is inserted into the position of the container denoted by the reference number 11, in the injection system (as described above and in FIG. 1), i.e. it is secured in the grip 4 and a plunger, such as the plunger 6 having notches or grooves on its piston 6a, is inserted into the container. When correctly inserted, the protruding members 40b fit into the notches or grooves of the piston. Thus, when the arms of the grip is pushed together, the plunger is pushed into the container and the material is pushed out of the container via the needle/cannula.

The materials used for the construction of the injection device or injection system should be any material that does not interact with the ceramic biomaterials, PMMA and other biomaterials, including solutions, to be injected. Said materials may also comprise inert particles.

Said materials should also be possible to sterilise by any of the sterilisation methods approved for sterilisation of medical devices, and which preferably can stand the radiation used in radiation treatment. Suitable materials would most likely be some sort of plastics or rubber, that may also be disposable. The material used for the extension tubing needs to be rigid and withstand the forces required injection without causing “after flow”.

The design of the could vary depending on design of container. For the PVP application the container will most likely be a 6 ml syringe.

The “grip” 4 for injecting ceramic paste is designed to allow injection of ceramic paste from a 1-10 ml syringe providing a power transition which is 3-5 times the normal hand power. This is an appropriate power ratio in order to reduce the resistance and enabling control of the injection, thus making it easy for a normal person.

The protruding members 40b (see FIG. 9) are uniquely designed to ensure that the teeth of the bridge joining said members 40b are constantly getting a new grip each time the arms 40a are pushed together. This constantly places the bridge into a new position, allowing further injection. In addition, said construction directs the feeding forces straight forward, in the direction of the injection, thereby avoiding the teeth of the bridge to jump out of position in the grooves/notches on the piston 6a. The teeth of said piston are large enough to secure a good grip and thus smooth injection. The teeth are also frequent enough to allow injection in sufficient small volumes at a time, while at the same time being possible to rationally produce using industrial tools. This also applies for the teeth of the bridge.

According to a basic embodiment of the method of injection using the injector device according to the present invention, said method comprises the steps of:

mixing the ceramic powder and liquid;

shaking said mixture;

feeding the ceramic composition or material out of the injector.

When injecting using the injector device according to embodiment 1A, i.e. the single-chamber container, the ceramic material is mixed by mixing the ceramic powder and liquid in advance. The ceramic powder and liquid originate from separate containers, for example enclosed and stored in blister packs or similar primary packages up to the moment they are to be mixed. The ceramic mixture is transferred from a mixing device and filled into the single-chamber, single-plunger container used, by any suitable means. The filled container is then mounted in the injector device. The injector is connected to an injection needle/cannula. The injector device may also be used in combination with an extension tube, said tube being connected to an injection needle/cannula. The injection is performed by pushing the arms of the grip together such that the feeding means feeds the plunger into the container and the ceramic composition or material out of the container.

When injecting using the injector device according to embodiment 1B, i.e. the dual-chamber container. The dual-chamber containers may be provided as syringes pre-filled with the ceramic powder liquid separated by a membrane. The method starts by pushing the inner plunger into the central bore of the outer plunger. This may be made even if the container is enclosed in a blister pack or a similar primary package. The liquid inside the distal part of the central bore which is not occupied by the inner plunger initially forms a part of the liquid chamber, working as a hydraulic means, when the inner plunger is pushed into the outer plunger, thus pushing the liquid with power leverage into the liquid chamber, breaking the membrane and bringing the ceramic powder into contact with the liquid. (see FIGS. 3-4). Thereafter the container is shaken in a shaking/vibrating apparatus, such that a ceramic paste or putty is formed. The container is then mounted into the injector device. The outer plunger is then screwed into the container, whereby the central bore/liquid chamber is charged with ceramic material. At the same time, the notches or grooves of the piston of the inner plunger are exposed. When said notches or grooves are exposed, the arms of the grip is pushed together such that the protruding member interacts with the notches or grooves of the inner plunger and pushes it into the central bore of the outer plunger, thereby, with enhanced power leverage, pushing the paste or putty out of the container via the outlet. This creates a power leverage which enables highly viscous substances, such as ceramic materials, to be injected.

The injector devices described in embodiments 1A and 1B may also be used in combination with an extension tube and/or cannula, as described above. The use of such an extension does not change the way the methods of injection is performed.

In the case of embodiments 2A and 2B, the single-chamber/dual chamber container is mixed and shaken as described above. The container is then mounted in the hydraulic extension system, in the position of container 11, as depicted in FIG. 6. The injection is then performed as described for embodiment 1A.

The tubing of the hydraulic means needs to be non-flexible, in the sense that diameter does not change due to an increased internal pressure, in order to cope with the forces required for the injection and still result in a firm and robust injection mode. However, the tubing may be bendable, in order to facilitate the handling of the injection system.

The membrane mentioned above may be breakable by vigorous shaking or a significant increase of the pressure inside said container, caused by the force of an internal plunger or its action.

The injector device or injection system, as well as the filling and transfer device may be disposable.

1. Mixing and injection device (1) for injecting an injectable biomaterial, comprising a container (2), for holding a first component and a second component initiating curing of the first component, separated from the first component, a grip (4), and means for feeding the biomaterial out of the container.

2. Mixing and injection device according to claim 1, wherein said grip (4) comprises one or more arms (40a).

3. Mixing and injection device according to claim 2, wherein said one or more arms (40a) comprises a protruding member (40b) forming an angle of less than 90 degrees to said one or more arms (40a).

4. Mixing and injection device according to claim 3, wherein said feeding means are comprised of said one or more arms (40a) and said protruding member (40b).

5. Mixing and injection device according to claim 4, wherein said container (2) is arranged and secured by a fastening means in the grip (4).

6. Mixing and injection device according to claim 5, wherein said container (2) is a single-chamber container (2).

7. Mixing and injection device according to claim 6, wherein said single-chamber container (2) comprises the first component.

8. Mixing and injection device according to claim 6, wherein said single-chamber container (2) comprises a mixture of the first component and the second component, said second component being separated from the first component.

9. Mixing and injection device according to claim 6, wherein said container (2) comprises a single-plunger (6) comprising a piston (6a) provided with notches or grooves.

10. Mixing and injection device according to claim 9, wherein said one or more arms (40a) each comprises two protruding members (40b) integrally joined by a dented bridge, the teeth of which grip into said notches or grooves.

11. Mixing and injection device according to claim 10, further comprising an extension tube (7) secured to the distal part of the container by the fastening means (3) or another fastening means, and optionally comprising a cannula, at the distal part of the extension tube (7).

12. Mixing and injection device according to claim 5, wherein said container (2) is a dual-chamber container (20), for holding the first component in a chamber (20a) and the second component in a chamber (20b), said chambers being separated by a membrane (20c).

13. Mixing and injection device according to claim 12, wherein said dual-chamber container (20) holds the first component in a chamber (20a) and the second component in a chamber (20b), said chambers being separated by a membrane (20c).

14. Mixing and injection device according to claim 12, wherein the injector device comprises two plungers, an outer plunger (50) threaded into the proximal end of the container (20) and having a central bore (50a), and an inner plunger (60) partially arranged inside said central bore (50a), wherein the distal part of the central bore which is not occupied by the inner plunger initially forms a part of the second component chamber (20b).

15. Mixing and injection device according to claim 13, further comprising an extension tube (7) secured to the distal part of the container by the fastening means (3) or another fastening means, and optionally comprising a cannula, at the distal part of the extension tube (7).

16. Mixing and injection device according to claim 4, wherein the container is separated from the grip (4) and feeding means by a hydraulic extension means (7).

17. Mixing and injection device according to claim 16, wherein the grip (4) is one part of the hydraulic extension means (7).

18. Mixing and injection device according to claim 17, wherein the hydraulic extension means (7) is connected to the distal end of a liquid-filled container (8), and being in open contact with each other, said container (8) being arranged in the grip (4), the hydraulic extension means is in the distal end connected to a second container (9) secured in a coupling means (10), which at its distal part is connected to a third container (11), the containers (9,11) and coupling means (10) form an open container into which a double-sided plunger (13) is positioned, said double-sided plunger (13) being in operative connection with the plunger of said container, and the distal part of the third container 11 is optionally adapted to be secured in a cannula 12 by a connector.

19. Mixing and injection device according to claim 18, wherein said container (11) is a single-chamber container (2).

20. Mixing and injection device according to claim 19, wherein said single-chamber container (2) comprises the first component.

21. Mixing and injection device according to claim 19, wherein said single-chamber container (2) comprises the first component and the second component, said second component being separated from the first component.

22. Mixing and injection device according to claim 19, wherein said container (11) comprises a single-plunger (6).

23. Mixing and injection device according to claim 18, wherein said container (11) is a dual-chamber container (20), wherein the first component located in a chamber (20a) and the second component located in a chamber (20b) are separated by a membrane (20c).

24. Mixing and injection device according to claim 23, wherein the injector device comprises two plungers, an outer plunger (50) threaded into the proximal end of the container (11) and having a central bore (50a), and an inner plunger (60) partially arranged inside said central bore (50a), wherein the distal part of the central bore which is not occupied by the inner plunger initially forms a part of the second component chamber (20b).

25. Mixing and injection device according to claim 1, wherein the injector device comprises a dual-chamber container (20), said container comprising a first component chamber having an outlet and a second component chamber (20b), said chambers being separated by a membrane (20c), said container being arranged and secured by fastening means at the distal end of a grip (40) having one or more arms (40a) provided at the proximal end with a protruding member (40b) forming an angle of less than 90 degrees to the arms of the grip, an outer plunger (50) threaded into the proximal end of the container (20) and having a central bore (50a) inside which an inner plunger (60) is partially arranged, the piston (60a) of which comprising notches or grooves, the distal part of the central bore which is not occupied by the inner plunger initially forms a part of the second component chamber (20b), and optionally the entire injector device is enclosed in a blister pack.

26. Mixing and injection device according to claim 1, wherein the injector device comprises a comprises a single-chamber container (2), for holding a biomaterial, and having a distally located outlet (2a), said container being arranged and secured by fastening means in the grip (4) having one or more arms (40a) provided at the proximal end with a protruding member (40b) forming an angle of less than 90 degrees to the arms of the grip, an inner plunger (6) partially arranged in the chamber, the piston (6a) of which comprising notches or grooves, and optionally the entire injector device is enclosed in a blister pack.

27. Method of injecting a biomaterial using the mixing and injection device according to claim 10, comprising the steps of:
mixing the a component and a second component, initiating curing of the first component, until a paste or putty is formed,
transferring the formed paste or putty to the single-chamber container (2);
arranging the single-chamber container in the grip (4), between the arms (40a) and securing the container in the grip by fastening means;
pushing the arms (40a) of the grip together such that the protruding member (40b) interacts with the notches or grooves of the plunger (6) and pushes the plunger into the container, thereby pushing the paste or putty out of the container via the outlet (2aa).

28. Method of injecting a biomaterial using the mixing and injection device according to claim 14, comprising the steps of:
pushing the inner plunger (6) into the central bore (50a) of the outer plunger (50), thereby increasing the pressure inside the second component chamber (20b) and breaking the membrane (20c), such that the first component is brought into contact with the second component;
shaking the injector device (1) in a shaking or vibrating apparatus until a paste or putty is formed;
screwing the outer plunger (50) into the proximal part of the container (20), thereby loading the bore (50a) with the paste or putty, while also exposing the notches or grooves of the inner plunger (60);
pushing the arms (40a) of the grip together such that the protruding member (40b) interacts with the notches or grooves of the inner plunger and pushes it into the central bore (50a) of the outer plunger, thereby pushing the paste or putty out of the container via the outlet (2aa).

29. Method of injecting a biomaterial using the mixing and injection device according to claim 22, comprising the steps of:
mixing a first component and a second component, initiating curing of the first component, until a paste or putty is formed;
transferring the formed paste or putty to the single-chamber container (2);
arranging the container (2) in the injection system and securing it between the coupling means (10) and the cannula (12):
pushing the arms (40a) of the grip (4) together such that the protruding member (40b) interacts with the notches or grooves of the plunger (6) and pushes the plunger into the container (8), thereby pushing the paste or putty out of the container (2) and cannula (12).

30. Method of injecting a biomaterial using the mixing and injection device according to claim 25, comprising the steps of:
pushing the inner plunger (6) into the central bore (50a) of the outer plunger (50) of the container (20), thereby increasing the pressure inside the second component chamber (20b) and breaking the membrane (20c), such that the first component is brought into contact with the second component;
shaking the injector device (1) in a shaking apparatus until a paste or putty is formed;
arranging the container (20) in the injection system and secure it between the coupling means (10) and the cannula (12):
pushing the arms (40a) of the grip (4) together such that the protruding member (40b) interacts with the notches or grooves of the plunger (6) and pushes the plunger into the container (8), thereby pushing the paste or putty out of the container (20) and cannula (12).

31. A transfer device (15) for transferring a biomaterial to the mixing and injection device defined in claim 1, wherein said transfer device comprises a capsule holder (16), a capsule (17) for holding a biomaterial, a connector (18), which is connected to the distal end of the capsule holder (16) as a separate part, or forms an integral end part of said capsule holder, wherein said connector (18) connects the capsule (17) at it proximal end and a single-chamber container (19) at its distal end, such that an open connection is formed between said capsule and said single-chamber container, and a screw or crank (21) threaded into the proximal part of said capsule holder (16).

32. The transfer device according to claim 31, wherein said capsule (17) is compressible, or has a bottom that can be pushed inside the body of the capsule or said capsule by said screw or crank (21), when said screw or crank is screwed into said thread, thus pushing the biomaterial in the capsule (17) into the single-chamber container (19).

33. The transfer device according to claim 32, wherein said connector (18), at its distal part, also connects a protective cover (20), wherein said protective cover covers the open connection between said capsule (17) and said single-chamber container (19).

34. The transfer device according to claim 33, wherein said protective cover (20) is removable together with the single-chamber container (19).
